(19) United States
(12) Patent Application Publication
(10) Pub. No.: US 2024/0398883 A1
(43) Pub. Date: Dec. 5, 2024

(54) CHINESE MEDICINE COMPOSITION FOR TREATING LUNG CANCER AND PREPARATION METHOD THEREOF

(71) Applicant: YU-TE LIU, Taichung City (TW)

(72) Inventors: YU-TE LIU, Taichung City (TW); Tsai-Ching HSU, Taichung City (TW); Bor-Show TZANG, Taichung City (TW)

(73) Assignee: YU-TE LIU, Taichung City (TW)

(21) Appl. No.: 18/241,480

(22) Filed: Sep. 1, 2023

(30) Foreign Application Priority Data
May 30, 2023 (TW) 112120110

(51) Int. Cl. A61K 36/232; A61K 33/04; A61K 36/31; A61K 36/539; A61K 36/736; A61P 35/00

(52) U.S. Cl. CPC A61K 36/232; A61K 33/04; A61K 36/31; A61K 36/539; A61K 36/736; A61P 35/00

(57) ABSTRACT

A Chinese medicine composition for treating lung cancer and a preparation method thereof. The Chinese medicine combination includes Bai Zhi (Angelica dahurica), Huang Qin (Scutellaria baicalensis), Ting Li Zi (Lepidium apetalum), Xing Ren (Apricot kernel), and Mang Xiao (Mirabilite). The content of each ingredients is measured by weight, with Bai Zhi ranging from 3 to 30 parts by weight, Huang Qin ranging from 3 to 30 parts by weight, Ting Li Zi ranging from 8 to 35 parts by weight, Xing Ren ranging from 5 to 30 parts by weight, and Mang Xiao ranging from 0.3 to 5 parts by weight. Cellular experiments have confirmed that when the Chinese medicine composition at an effective dosage of 1 to 100 μg/ml is administered to the lung cancer cells in vitro, LC3-I/LC3-II proteins are induced to express, thereby triggering autophagy in the lung cancer cells to inhibit or reduce cell activity.

### BACKGROUND OF THE INVENTION

### Technical Field

The present invention relates generally to a traditional Chinese medicine for treating lung cancer, and more particularly to a Chinese medicine composition for treating lung cancer and a preparation method thereof, which could inhibit the activity of lung cancer cells.

### Description of Related Art

Lung cancer has a high mortality rate due to the difficulty of early diagnosis. By the time patients are found to have lung cancer cells, most of the cancer cells have already metastasized, making the treatment effect limited. Currently, common treatment methods for lung cancer include lung function resection surgery, radiation therapy, and chemotherapy. Among them, chemotherapy involves using targeted drugs to inhibit the growth of lung cancer cells, slow down their spread, and prevent cancer recurrence.

However, the targeted drugs currently used in chemotherapy have significant side effects on the human body. In order to improve the side effects of cancer treatment, some related companies are developing natural compounds such as traditional Chinese medicine. Many studies have indicated that specific traditional Chinese medicines can be used as adjunctive therapy for the treatment of advanced non-small cell lung cancer (NSCLC).

For example, Scutellaria radix (Huang Qin) and Angelica dahurica (Bai Zhi) have shown certain efficacy in cancer treatment in many research studies. Simply put. Scutellaria radix not only promotes the production of immunoglobulins in patients to enhance their immune system but its extract has been proven to have cytotoxic effects on non-small cell lung cancer cells, significantly inhibiting the metastatic ability of non-small cell lung cancer cells. Angelica dahurica contains effective ingredients such as furanocoumarins, which can induce apoptosis in human lung cancer cells and indeed have therapeutic effects against cancer.

However, although the aforementioned research has demonstrated the efficacy of specific natural components in traditional Chinese medicine in inhibiting lung cancer cells, individual herbs still have limited effectiveness in treating lung cancer. Therefore, the selection of Chinese herbs to prepare a Chinese medicine composition for effectively treating lung cancer is the problem that needs to be solved in the present invention.

### BRIEF SUMMARY OF THE INVENTION

In view of the above, the primary objective of the present invention is to provide a Chinese medicine composition for treating lung cancer and a and preparation method thereof, wherein the Chinese Medicine composition could reduce or inhibit the activity of the lung cancer cells. The Chinese medicine composition includes Angelica dahurica, Scutellaria baicalensis, Lepidium apetalum, apricot kernel, and mirabilite, which could effectively induce the autophagy of the non Non-Small Cell Lung Cancer (NSCLC) cells and Small Cell Lung Cancer (SCLC) cells, thereby inhibiting and reducing the viable cancer cells of either NSCLC or SCLC.

The present inventive subject matter provides a Chinese medicine composition including Angelica dahurica ranging from 3 to 30 parts by weight, Scutellaria baicalensis ranging from 3 to 30 parts by weight, Lepidium apetalum ranging from 8 to 35 parts by weight, apricot kernel ranging from 5 to 30 parts by weight, and mirabilite ranging from 0.3 to 5 parts by weight.

In an embodiment, a sum of a content of Angelica dahurica and a content of the Scutellaria baicalensis is a first portion, and a sum of a content of Lepidium apetalum, a content of apricot kernel, and a content of mirabilite is a second portion. The first portion is greater than the second portion.

In an embodiment, Angelica dahurica ranges from 5 to 10 parts by weight. Scutellaria baicalensis ranges from 10 to 15 parts by weight. Lepidium apetalum ranges from 10 to 15 parts by weight. Apricot kernel ranges from 8 to 12 parts by weight. Mirabilite ranges from 0.5 to 1.5 parts by weight.

In an embodiment, Angelica dahurica ranges from 8 to 10 parts by weight. Scutellaria baicalensis ranges from 13 to 15 parts by weight. Lepidium apetalum ranges from 10 to 12 parts by weight. Apricot kernel ranges from 8 to 10 parts by weight. Mirabilite ranges from 1 to 1.5 parts by weight.

In an embodiment, a sum of a content of Scutellaria baicalensis and a content of the Lepidium apetalum is a third portion. A sum of a content of Angelica dahurica, a content of apricot kernel, and a content of mirabilite is a fourth portion. The third portion is greater than the fourth portion.

In an embodiment, Angelica dahurica ranges from 5 to 10 parts by weight. Scutellaria baicalensis ranges from 20 to 30 parts by weight. Lepidium apetalum ranges from 20 to 30 parts by weight. Apricot kernel ranges from 8 to 12 parts by weight. Mirabilite ranges from 0.5 to 1.5 parts by weight.

In an embodiment, a sum of a content of Angelica dahurica and a content of the Scutellaria baicalensis is a first portion. A sum of a content of Lepidium apetalum, a content of apricot kernel, and a content of mirabilite is a second portion. The second portion is greater than the first portion.

In an embodiment, Angelica dahurica ranges from 15 to 25 parts by weight. Scutellaria baicalensis ranges from 10 to 15 parts by weight. Lepidium apetalum ranges from 20 to 30 parts by weight. Apricot kernel ranges from 15 to 25 parts by weight. Mirabilite ranges from 2 to 4 parts by weight.

The present inventive subject matter provides an usage of the Chinese medicine composition as claimed in claim 1 for inhibiting or reducing an activation of lung cancer cells, wherein when the Chinese medicine composition at an effective dose of 1 μg/ml to 100 μg/ml administrates to lung cancer cells, the LC3-I/LC3-II protein is induced to express, leading to autophage of lung cancer cells.

In an embodiment, the effective dose of said Chinese medicine composition is in a range of 25 μg/ml to 100 μg/ml, and said lung cancer cells include Non-Small Cell Lung Cancer (NSCLC) cells and/or Small Cell Lung Cancer (SCLC) cells.

Through cellular experiments, it has been confirmed that the pharmacological interactions between the ingredients of the Chinese medicine composition, including Angelica dahurica, Scutellaria baicalensis, Lepidium apetalum, apricot kernel, and mirabilite, could effectively suppress or reduce the viability of NSCLC cells and SCLC cells within a short period of time. Additionally, the Chinese medicine composition, under effective dosage conditions of 25-100 μg/ml, can significantly induce the expression of LC3-I/LC3-II proteins in non-small cell lung cancer cells and small cell lung cancer cells, which could trigger autophagy in the lung cancer cells, thereby achieving a therapeutic effect or improvement in the treatment of lung cancer.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1A is a bar chart, showing the cell viability of the lung adenocarcinoma cells (H460) that are respectively treated with Huang Qin (Scutellaria baicalensis) at different effective doses, including 0 μg/ml, 1 μg/ml, 5 μg/ml, 25 μg/ml, 50 μg/ml, and 100 μg/ml;

FIG. 1B is a protein electrophoresis gel image, showing the LC3-I/LC3-II proteins expression of the lung adenocarcinoma cells (H460) that are respectively treated with Huang Qin (Scutellaria baicalensis) at different effective doses, including 0 μg/ml, 1 μg/ml, 5 μg/ml, 25 μg/ml, 50 μg/ml, and 100 μg/ml;

FIG. 2A is a bar chart, showing the cell viability of the lung adenocarcinoma cells (H460) that are respectively treated with Bai Zhi (Angelica dahurica) at different effective doses, including 0 μg/ml, 1 μg/ml, 5 mg/ml, 25 mg/ml, 50 μg/ml, and 100 mg/ml;

FIG. 2B is a protein electrophoresis gel image, showing the LC3-I/LC3-II proteins expression of the lung adenocarcinoma cells (H460) that are respectively treated with Bai Zhi (Angelica dahurica) at different effective doses, including 0 μg/ml, 1 μg/ml, 5 μg/ml, 25 μg/ml, 50 μg/ml, and 100 μg/ml;

FIG. 3A is a bar chart, showing the cell viability of the lung adenocarcinoma cells (H460) that are respectively treated with the mixture of Huang Qin (Scutellaria baicalensis) and Bai Zhi (Angelica dahurica) at different effective doses, including 0 μg/ml, 1 μg/ml, 5 μg/ml, 25 μg/ml, 50 μg/ml, and 100 μg/ml;

FIG. 3B is a protein electrophoresis gel image, showing the LC3-I/LC3-II proteins expression of the lung adenocarcinoma cells (H460) that are respectively treated with the mixture of Huang Qin (Scutellaria baicalensis) and Bai Zhi (Angelica dahurica) at different effective doses, including 0 μg/ml, 1 μg/ml, 5 μg/ml, 25 μg/ml, 50 μg/ml, and 100 μg/ml;

FIG. 4A is a bar chart, showing the cell viability of the lung adenocarcinoma cells (H460) that are respectively treated with the Chinese medicine composition of a first embodiment at different effective doses, including 0 μg/ml, 1 μg/ml, 5 μg/ml, 25 μg/ml, 50 μg/ml, and 100 μg/ml;

FIG. 4B is a protein electrophoresis gel image, showing the LC3-I/LC3-II proteins expression of the lung adenocarcinoma cells (H460) that are respectively treated with the Chinese medicine composition of the first embodiment at different effective doses, including 0 μg/ml, 1 μg/ml, 5 μg/ml, 25 μg/ml, 50 μg/ml, and 100 μg/ml;

FIG. 5A is a bar chart, showing the cell viability of the SCLC cells (H1355) that are respectively treated with Huang Qin (Scutellaria baicalensis) at different effective doses, including 0 μg/ml, 1 μg/ml, 5 μg/ml, 25 μg/ml, 50 μg/ml, and 100 μg/ml;

FIG. 5B is a protein electrophoresis gel image, showing the LC3-I/LC3-II proteins expression of the SCLC cells (H1355) that are respectively treated with Huang Qin (Scutellaria baicalensis) at different effective doses, including 0 μg/ml, 1 μg/ml, 5 μg/ml, 25 μg/ml, 50 μg/ml, and 100 μg/ml;

FIG. 6A is a bar chart, showing the cell viability of the SCLC cells (H1355) that are respectively treated with Bai Zhi (Angelica dahurica) at different effective doses, including 0 μg/ml, 1 μg/ml, 5 μg/ml, 25 μg/ml, 50 μg/ml, and 100 μg/ml;

FIG. 6B is a protein electrophoresis gel image, showing the LC3-I/LC3-II proteins expression of the SCLC cells (H1355) that are respectively treated with Bai Zhi (Angelica dahurica) at different effective doses, including 0 μg/ml, 1 μg/ml, 5 μg/ml, 25 μg/ml, 50 μg/ml, and 100 μg/ml;

FIG. 7A is a bar chart, showing the cell viability of the SCLC cells (H1355) that is respectively treated with the mixture of Huang Qin (Scutellaria baicalensis) and Bai Zhi (Angelica dahurica) at different effective doses, including 0 μg/ml, 1 μg/ml, 5 μg/ml, 25 μg/ml, 50 μg/ml, and 100 μg/ml;

FIG. 7B is a protein electrophoresis gel image, showing the LC3-I/LC3-II proteins expression of the SCLC cells (H1355) that are respectively treated with the mixture of Huang Qin (Scutellaria baicalensis) and Bai Zhi (Angelica dahurica) at different effective doses, including 0 μg/ml, 1 μg/ml, 5 μg/ml, 25 μg/ml, 50 μg/ml, and 100 μg/ml;

FIG. 8A is a bar chart, showing the cell viability of the SCLC cells (H1355) that are respectively treated with the Chinese medicine composition of a first embodiment at different effective doses, including 0 μg/ml, 1 μg/ml, 5 μg/ml, 25 μg/ml, 50 μg/ml, and 100 μg/ml; and

FIG. 8B is a protein electrophoresis gel image, showing the LC3-I/LC3-II proteins expression of the SCLC cells (H1355) that are respectively treated with the Chinese medicine composition of the first embodiment at different effective doses, including 0 μg/ml, 1 μg/ml, 5 μg/ml, 25 μg/ml, 50 μg/ml, and 100 μg/ml.

### DETAILED DESCRIPTION OF THE INVENTION

A Chinese medicine composition for treating lung cancer includes Angelica dahurica, Scutellaria baicalensis, Lepidium apetalum, apricot kernel, and mirabilite, wherein the Angelica dahurica, Scutellaria baicalensis, Lepidium apetalum, apricot kernel, and mirabilite are obtained through extraction of corresponding natural plants.

The pharmacological characteristics of each of the ingredients in the Chinese medicine composition are described in detail below:

Angelica dahurica (Bai Zhi): the active components of Angelica dahurica include angelicin, coumarin, and furanocoumarins, which have antipyretic, analgesic, antibacterial, anti-inflammatory, and antioxidant effects. Additionally, the components shows inhibitory effects on bacteria, fungi, and yeast.

Scutellaria baicalensis (Huang Qin): The active components of Scutellaria baicalensis include acacetin, apigenin, baicalin, chrysin, wogonin, and so on. Each of the active components has specific therapeutic effects, such as anticancer, hepatoprotective (liver-protecting), anti-cardiovascular disease, antiallergic, and anti-inflammatory properties, which is also beneficial in tumor suppression.

Lepidium apetalum (Ting Li Zi): The main active components of Lepidium apetalum include isothiocyanates, glycosides, as well as flavonoids. The thiocyanates and their glucosinolates are common components in Brassicaceae plants and have antitussive and anti-asthmatic effects.

Apricot kernel (Xing Ren): The effective components of apricot kernel include Amygdalin, Amygdalic Acid, various free amino acids, Chlorogenic Acid, and Benzoic Acid. After oral intake, Amygdalin is broken down to produce a small amount of hydrogen cyanide, which could inhibit the cough center, leading to antitussive and anti-asthmatic effects. Apricot kernel is clinically used for various cough and asthma treatments.

Mirabilite (Mang Xiao): Pharmacological effects of Mirabilite include heat-clearing and bowel-cleansing, moistening dryness and softening hardness, clearing heat and reducing swelling, treating swollen and painful throat, mouth and tongue sores, swollen and painful gums, red and swollen eyes, and toxic heat-related conditions.

The formula of the Chinese medicine composition could be adjusted based on the extent of metastasis of the cancer cells. In other words, the ratio of ingredients of the Chinese medicine composition, including Angelica dahurica, Scutellaria baicalensis, Lepidium apetalum, apricot kernel, and mirabilite, could be adjusted according to different metastasis stages. The content of each ingredient of the Chinese medicine composition is measured by weight. The Chinese medicine composition includes Angelica dahurica ranging from 3 to 30 parts by weight, Scutellaria baicalensis ranging from 3 to 30 parts by weight, Lepidium apetalum ranging from 8 to 35 parts by weight, apricot kernel ranging from 5 to 30 parts by weight, and mirabilite ranging from 0.3 to 5 parts by weight.

A formula of the chinese medicine composition of a first embodiment is described as below. A sum of a content of Angelica dahurica and a content of the Scutellaria baicalensis is a first portion. A sum of a content of Lepidium apetalum, a content of apricot kernel, and a content of mirabilite is a second portion. The first portion is greater than the second portion. More specifically, the Chinese medicine composition of the first embodiment includes Angelica dahurica ranging from 5 to 10 parts by weight, Scutellaria baicalensis ranging from 10 to 15 parts by weight, Lepidium apetalum ranging from 10 to 15 parts by weight, apricot kernel ranging from 8 to 12 parts by weight, and mirabilite ranging from 0.5 to 1.5 parts by weight. Preferably, the Chinese medicine composition includes Angelica dahurica ranging from 8 to 10 parts by weight, Scutellaria baicalensis ranging from 13 to 15 parts by weight, Lepidium apetalum ranging from 10 to 12 parts by weight, apricot kernel ranging from 8 to 10 parts by weight, and mirabilite ranging from 1 to 1.5 parts by weight. In clinical practice of Chinese medicine, when the lung cancer cells do not spread to other organs, the Chinese medicine composition of the first embodiment, which is made of Angelica dahurica, Scutellaria baicalensis, Lepidium apetalum, apricot kernel, and mirabilite could in specific parts by weights, could effectively suppress or reduce the viability of the lung cancer cells within a short time.

A formula of the Chinese medicine composition of a second embodiment is similar to the formula of the Chinese medicine composition of the first embodiment, except the content of Scutellaria baicalensis and Lepidium apetalum are increased. The formula of the Chinese medicine composition of the second embodiment is described as below. A sum of a content of Scutellaria baicalensis and a content of the Lepidium apetalum is a third portion. A sum of a content of Angelica dahurica, a content of apricot kernel, and a content of mirabilite is a fourth portion. The third portion is greater than the fourth portion. More specifically, the Chinese medicine composition of the second embodiment includes Angelica dahurica ranging from 5 to 10 parts by weight. Scutellaria baicalensis ranging from 20 to 30 parts by weight, Lepidium apetalum ranging from 20 to 30 parts by weight, apricot kernel ranging from 8 to 12 parts by weight, and mirabilite ranging from 0.5 to 1.5 parts by weight. In clinical practice of Chinese medicine, when the lung cancer cells do not spread to other organs and patient with pulmonary edema present, the Chinese medicine composition of the second embodiment could not only suppress and reduce the viability of the cancer cells, but also improve the symptoms of pulmonary edema.

A formula of the Chinese medicine composition of a third embodiment is similar to the formula of the Chinese medicine composition of the first embodiment, except the content of Angelica dahurica. Lepidium apetalum, apricot kernel, and mirabilite are increased. The formula of the Chinese medicine composition of the third embodiment is described as below. The second portion, the sum of the content of Lepidium apetalum, the content of apricot kernel, and the content of mirabilite, is greater than the first portion, the sum of the content of Angelica dahurica and the content of Scutellaria baicalensis. More specifically, the Chinese medicine composition of the third embodiment includes Angelica dahurica ranging from 15 to 25 parts by weight, Scutellaria baicalensis ranging from 10 to 15 parts by weight, Lepidium apetalum ranging from 20 to 30 parts by weight, apricot kernel ranging from 15 to 25 parts by weight, and mirabilite ranging from 2 to 4 parts by weight. In clinical practice of Chinese medicine, when the lung cancer cells spread to other organs, by regularly intaking the chinese medicine composition of the third embodiment, the metastasis of the cancer cells could be effectively controlled or improved.

Additionally, through the cellular experiments, the Chinese medicine composition of each of the first embodiment to the third embodiment is proved to be effective to inhibit or reduce the viability of the lung cancer cells. During the cell experiments, an effective dose of the Chinese medicine composition ranging from 1 μg/ml to 100 μg/ml is administrated to the lung cancer cells. The cellular experiments confirm that the effective dose of the Chinese medicine composition could effectively induce the LC3-I/LC3-II proteins in NSCLC cells and SCLC cells to express, thereby triggering the autophagy of the lung cancer cells. Preferably, the effective dose of the Chinese medicine composition ranging from 25 μg/ml to 100 μg/ml could significantly induce the LC3-I/LC3-II proteins in NSCLC cells and SCLC cells to express, which significantly suppress or inhibit the viability of the lung cancer cells, thereby curing or improving the lung cancer.

In order to demonstrate the purpose, the features, and the effects of the present invention, the Chinese medicine composition of the first embodiment is used to conduct the following cellular experiments. The Chinese medicine composition of the first embodiment at various effective doses are respectively administrated to NSCLC cells and SCLC cells, wherein the NSCLC cells in the cellular experiments are lung adenocarcinoma cells (H460), and the SCLC cells in the cellular experiments are SCLC cells (H1355). The growth and the viability of the lung cancer cells in each experiments are analyzed, and the result could explain the chinese medicine composition is effective to inhibit or reduce the viability of the lung cancer cells.

In the following experimental examples, an applicable range of the present invention could be proved, and the skilled person in the art could properly adjusted the parameters by referring to the following experimental examples, wherein the applicable range which is properly adjusted by the skilled person in the art is still in the scope of the present invention.

There are three comparative examples (1-3) and an experimental example:

Comparative example 1: Scutellaria baicalensis at difference effective doses are administrated to the lung adenocarcinoma cells (H460) and SCLC cells (H1355), respectively, wherein the effective dose of Scutellaria baicalensis includes 0 μg/ml, 1 μg/ml. 5 μg/ml, 25 μg/ml, 50 μg/ml, and 100 μg/m.

Comparative example 2: Angelica dahurica at difference effective doses are administrated to the lung adenocarcinoma cells (H460) and SCLC cells (H1355), respectively, wherein the effective dose of Angelica dahurica includes 0 μg/ml, 1 μg/ml, 5 μg/ml, 25 μg/ml, 50 μg/ml, and 100 μg/m.

Comparative example 3: A mixture is mixed by Scutellaria baicalensis 1 parts by weight and Angelica dahurica 1 part by weight. The mixture of Scutellaria baicalensis and Angelica dahurica at difference effective doses are administrated to the lung adenocarcinoma cells (H460) and SCLC cells (H1355), respectively, wherein the effective dose of Angelica dahurica includes 0 μg/ml, 1 μg/ml, 5 μg/ml, 25 μg/ml, 50 μg/ml, and 100 μg/m.

Experimental example: The Chinese medicine composition includes Angelica dahurica 10 parts by weight, Scutellaria baicalensis 15 parts by weight, Lepidium apetalum 12 parts by weight, apricot kernel 10 parts by weight, and mirabilite 1.5 parts by weight. The Chinese medicine composition at difference effective doses are administrated to the lung adenocarcinoma cells (H460) and SCLC cells (H1355), respectively, wherein the effective dose of Angelica dahurica includes 0 μg/ml, 1 μg/ml, 5 μg/ml, 25 μg/ml, 50 μg/ml, and 100 μg/m.

### MTT Assay (for Assessing Cell Metabolic Activity)

The test includes the following steps: plate either lung adenocarcinoma cells (H460) or SCLC cells (H1355) at 5×10³ in each well of the 96-well microplate and incubate 24 hours; after lung adenocarcinoma cells (H460) or SCLC cells (H1355) are attached to the wells, remove the cell culture medium; add the cell culture medium with the material of each of the comparative examples 1-3 and the experimental example at different effective doses, including 1 μg/ml, 5 μg/ml, 25 μg/ml, 50 μg/ml, and 100 μg/m into each well of the microplate that has the lung adenocarcinoma cells (H460) and SCLC cells (H1355), and wait 1-2 days; remove the cell culture medium, and wash the microplate with phosphate buffered saline (PBS) two times; add 0.2 ml MTT culture medium into each well, and incubate in a constant temperature incubator for 2 hours; remove the MTT culture medium, and wash the microplate with PBS once; and add 0.2 ml DMSO to dissolve crystals, and analyze the microplate by utilizing an ELISA (Enzyme-linked Immuno-sorbent Assay) reader to measure the optical density (OD) at 570 nm, thereby calculating the relatively cell viability.

The well that is untreated by any material (namely, 0 μg/ml effective dose) is taken as a control group, wherein the absorbance value of the control group is quantified as 100%. The measured OD of each of the comparative examples 1-3 is divided by the absorbance value of the control group to obtain the relatively cell viability of either lung adenocarcinoma cells (H460) or SCLC cells (H1355). Similarly, the well that is untreated by any material (namely, 0 μg/ml effective dose) is taken as a control group, wherein the absorbance value of the control group is quantified as 100%. The measured OD of the experimental example is divided by the absorbance value of the control group to obtain the relatively cell viability of either lung adenocarcinoma cells (H460) or SCLC cells (H1355).

### Western Blot (for Assessing Autophagy)

Measure the antibodies targeting LC3-I, LC3-II, and B-Actin, which are related to autophagy. Firstly, the nitrocellulose filter membrane (NC) is incubated with the antibodies in phosphate-buffered saline (PBS) containing 2.5% bovine serum albumin (BSA) for 3 hours. After washing three times with PBS, horse-radish peroxidase (HRP) antibodies are added and further incubated for 1 hour. Finally, the antigen-antibody complexes are detected by using Immobilon Western HRP chemiluminescent reagent, and the protein blots are quantified using a densitometer (Appraise, Beckman-Coulter, Brea, California, USA).

More specifically, cellular autophagy is a physiological process within cells that primarily involves the breakdown of old proteins, malfunctioning organelles, and foreign microorganisms. During the process of autophagy, numerous proteins are involved in regulation. Microtubule-associated protein 1A/1B-light chain 3 (LC3), located in the cytoplasm, is often used as a fluorescent marker to indicate autophagy activation. When autophagy is activated, LC3 binds to the lipid phosphatidylethanolamine to form LC3-II, which then embeds into the membrane of autophagosomes. As a result, the level of LC3-II generated within the cells serves as an indicator of autophagy activation.

I. Discussion: viability and autophagy of lung adenocarcinoma cells (H460) treated by the material of each of the comparative examples 1-3 and the experimental example:

### 1. Test of Lung Adenocarcinoma Cells (H460) Viability:

Analyze the survival status of lung adenocarcinoma cells (H460) treated by the material of each of the comparative examples 1-3 and the experimental example. After the material of each of the comparative examples 1-3 and the experimental example in various effective doses are administrated to lung adenocarcinoma cells (H460), the lung adenocarcinoma cells (H460) viability on the first day and the second day after the treatment is measured.

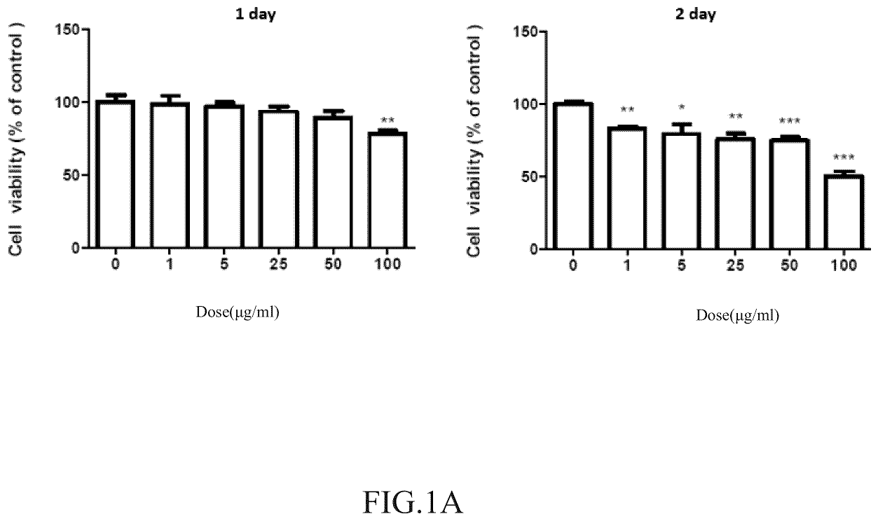

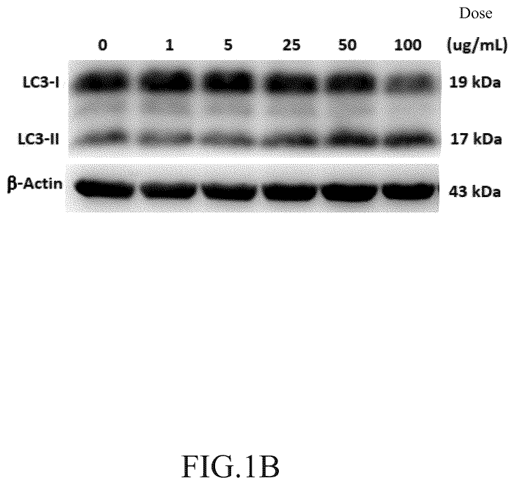

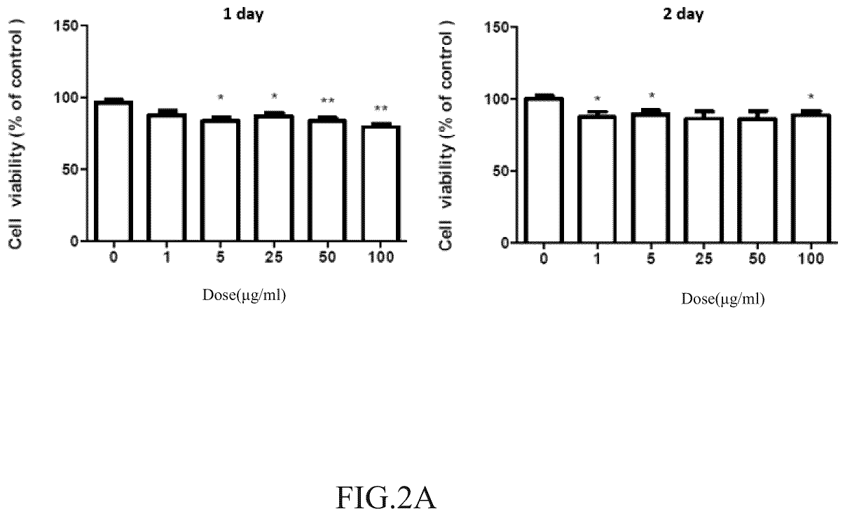

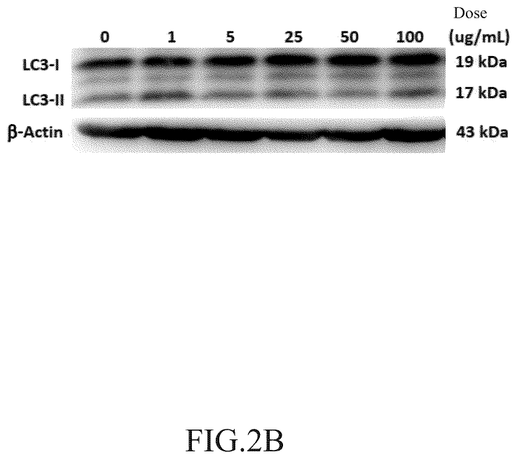

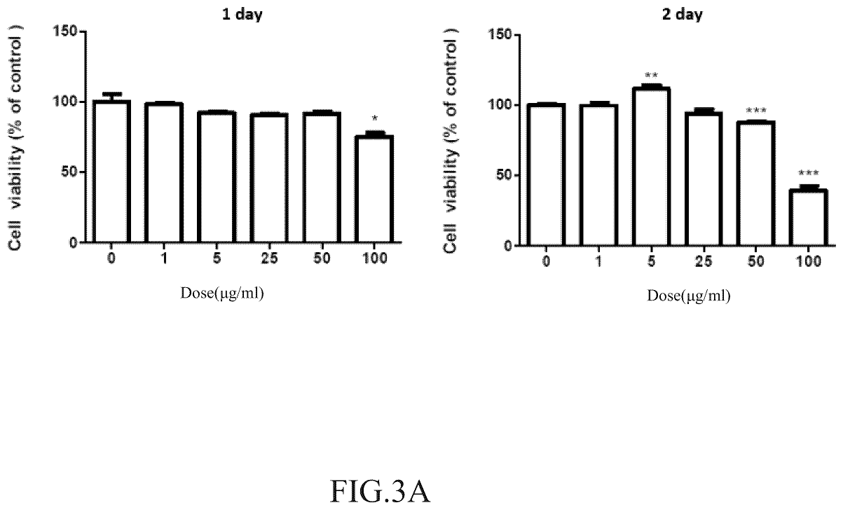

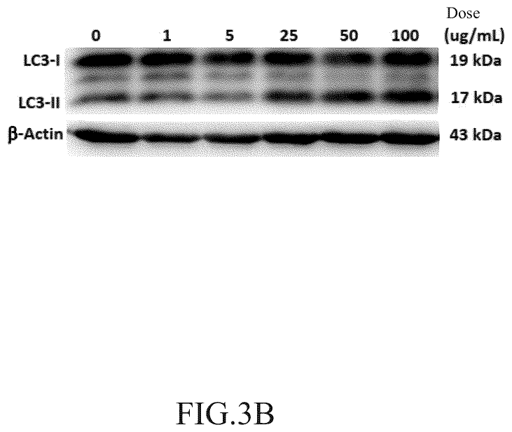

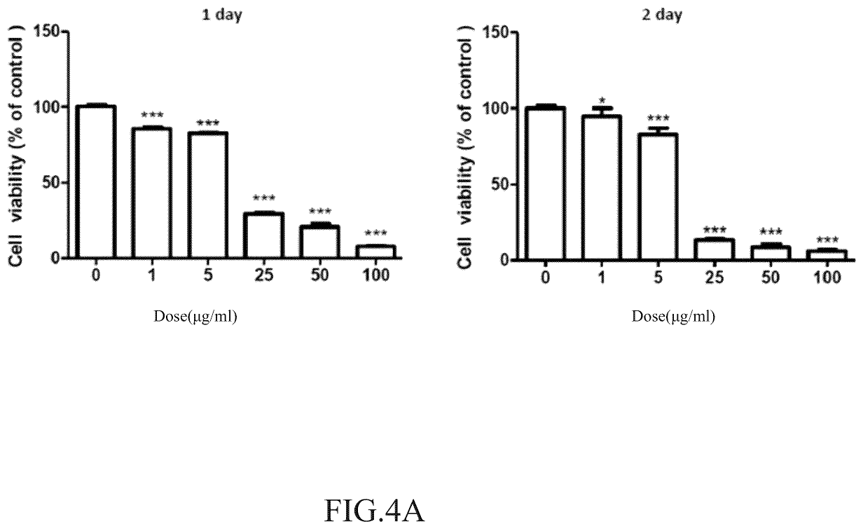

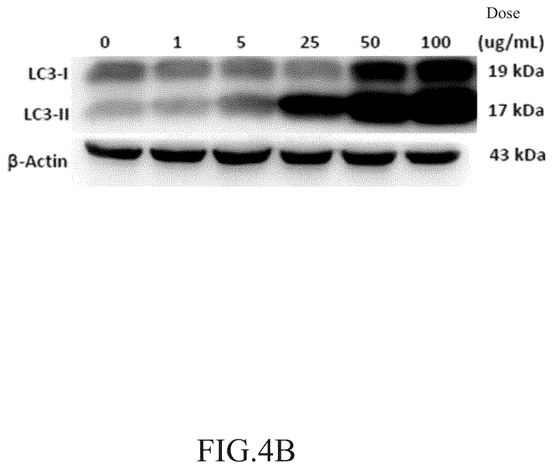

As illustrated in FIG. 1A, FIG. 2A, FIG. 3A, and FIG. 4A, FIG. 1A shows the lung adenocarcinoma cells (H460) viability on the first day and the second day after treated by the material of the comparative example 1 at different effective doses, including 0 μg/ml, 1 μg/ml, 5 μg/ml, 25 μg/ml, 50 μg/ml, and 100 μg/ml. FIG. 2A shows the lung adenocarcinoma cells (H460) viability on the first day and the second day after treated by the material of the comparative example 2 at different effective doses, including 0 μg/ml, 1 μg/ml, 5 μg/ml, 25 μg/ml, 50 μg/ml, and 100 μg/ml. FIG. 3A shows the lung adenocarcinoma cells (H460) viability on the first day and the second day after treated by the material of the comparative example 3 at different effective doses, including 0 μg/ml, 1 μg/ml, 5 μg/ml, 25 μg/ml, 50 μg/ml, and 100 μg/ml. FIG. 4A shows the lung adenocarcinoma cells (H460) viability on the first day and the second day after treated by the material of the experimental example at different effective doses, including 0 μg/ml, 1 μg/ml, 5 μg/ml, 25 μg/ml, 50 μg/ml, and 100 μg/ml.

In FIG. 1A, FIG. 2A, FIG. 3A, and FIG. 4A, the Standard Error (SE) of the test result of the comparative examples 1-3 and the experimental example is shown. The test result is obtained from three independent cellular experiments (n=3), wherein “*” indicates statistical significance (P<0.05) in the comparison of the test result of each of the comparative examples 1-3 and the experimental example.

As shown in FIG. 1A, in the result of the first day of the comparative example 1, the cell viabilities of lung adenocarcinoma cells (H460) treated by the material of the comparative example 1 at various effective doses do not significantly reduced. In the result of the second day of the comparative example 1, the cell viability of lung adenocarcinoma cells (H460) treated by the material of the comparative example 1 at the effective dose of 100 μg/ml is significantly reduced to approximately 50%. In other words, the Scutellaria baicalensis at the effective dose of 100 μg/ml could inhibit the activation of lung adenocarcinoma cells (H460) and reduce the amount of lung adenocarcinoma cells (H460).

As shown in FIG. 2A, in the result of either the first day or the second day of the comparative example 2, the cell viabilities of lung adenocarcinoma cells (H460) treated by the material of the comparative example 2 at various effective doses do not significantly reduced. In other words, merely using Angelica dahurica could not inhibit or reduce the viability or amount of lung adenocarcinoma cells (H460).

As shown in FIG. 3A, in the result of the first day of the comparative example 3, the cell viabilities of lung adenocarcinoma cells (H460) treated by the material of the comparative example 3 at various effective doses do not significantly reduced. In the result of the second day of the comparative example 3, the cell viability of lung adenocarcinoma cells (H460) treated by the material of the comparative example 3 at the effective dose of 100 μg/ml is significantly reduced to approximately 20%-30%. In other words, comparing to the comparative example 1 (only Scutellaria baicalensis), the mixture of Scutellaria baicalensis and the Angelica dahurica at the effective dose of 100 μg/ml could more effectively inhibit the activation of lung adenocarcinoma cells (H460) and reduce the amount of lung adenocarcinoma cells (H460).

As shown in FIG. 4A, in the result of the first day of the experimental example, the cell viabilities of lung adenocarcinoma cells (H460) treated by the material of the experimental example at various effective doses, including 25 μg/ml, 50 μg/ml, and 100 μg/m, are significantly reduced to a range of approximately 5%-20%. Moreover, the cell viability of lung adenocarcinoma cells (H460) treated by the material of the experimental example at effective dose of 100 μg/ml is significantly reduced. In the result of the second day of the experimental example, the cell viabilities of lung adenocarcinoma cells (H460) treated by the material of the experimental example at various effective doses, including 25 μg/ml, 50 μg/ml, and 100 μg/m, are further reduced to a range of approximately 1%-10%. Similarly, the cell viability of lung adenocarcinoma cells (H460) treated by the material of the experimental example at effective dose of 100 μg/ml is more significantly reduced. In other words, comparing to the comparative example 3 (namely, the mixture of Scutellaria baicalensis and Angelica dahurica), the Chinese medicine composition of the experimental example could more effectively inhibit the activation of lung adenocarcinoma cells (H460) and reduce the amount of lung adenocarcinoma cells (H460). Additionally, the the Chinese medicine composition of experimental example at the effective doses of 25 μg/ml, 50 μg/ml, or 100 μg/ml could reduce the amount of lung adenocarcinoma cells (H460) in the first day.

### 2. Autophage of Lung Adenocarcinoma Cells (H460):

The LC3-I/LC3-II protein expression of lung adenocarcinoma cells (H460) that is treated by each of the comparative examples 1-3 and the experimental example in various effective doses, including 1 μg/ml, 5 μg/ml, 25 μg/ml, 50 μg/ml, and 100 μg/ml, is analyzed. After lung adenocarcinoma cells (H460) is treated by each of the comparative examples 1-3 and the experimental example, the LC3-I/LC3-II protein expression of lung adenocarcinoma cells (H460) in each example is detected through the electrophoresis.

As illustrated in FIG. 1B, FIG. 2B, FIG. 3B, and FIG. 4B, FIG. 1B shows the LC3-I/LC3-II protein electrophoresis diagram of lung adenocarcinoma cells (H460) treated by the comparative example 1 at different effective doses, including 0 μg/ml, 1 μg/ml, 5 μg/ml, 25 μg/ml, 50 μg/ml, and 100 μg/m. FIG. 2B shows the LC3-I/LC3-II protein electrophoresis diagram of lung adenocarcinoma cells (H460) treated by the comparative example 2 at different effective doses, including 0 μg/ml, 1 μg/ml, 5 μg/ml, 25 μg/ml, 50 μg/ml, and 100 μg/m. FIG. 3B shows the LC3-I/LC3-II protein electrophoresis diagram of lung adenocarcinoma cells (H460) treated by the comparative example 3 at different effective doses, including 0 μg/ml, 1 μg/ml, 5 μg/ml, 25 μg/ml, 50 μg/ml, and 100 μg/m. FIG. 4B shows the LC3-I/LC3-II protein electrophoresis diagram of lung adenocarcinoma cells (H460) treated by the experimental example at different effective doses, including 0 μg/ml, 1 μg/ml, 5 μg/ml, 25 μg/ml, 50 μg/ml, and 100 μg/ml.

As illustrated in FIG. 1B, FIG. 2B, FIG. 3B, and FIG. 4B, the LC3-II protein expression of lung adenocarcinoma cells (H460) treated by the experimental example at the effective doses, including 25 μg/ml, 50 μg/ml, and 100 μg/ml, is obviously greater than that is treated by the comparative examples 1-3. In other words, the Chinese medicine composition of the experimental example at the effective dose of 25 μg/ml, 50 μg/ml, or 100 μg/ml could induce the LC3-I/LC3-II protein expression in lung adenocarcinoma cells (H460), leading to autophagy of lung adenocarcinoma cells (H460), thereby inhibiting and reducing the activation of lung adenocarcinoma cells (H460).

II. I. Discussion: viability and autophagy of SCLC cells (H1355) treated by the material of each of the comparative examples 1-3 and the experimental example:

### 1. Test of SCLC Cells (H1355) Viability:

Analyze the survival status of SCLC cells (H1355) treated by the material of each of the comparative examples 1-3 and the experimental example. After the material of each of the comparative examples 1-3 and the experimental example in various effective doses are administrated to SCLC cells (H1355), the SCLC cells (H1355) viability on the first day and the second day after the treatment is measured.

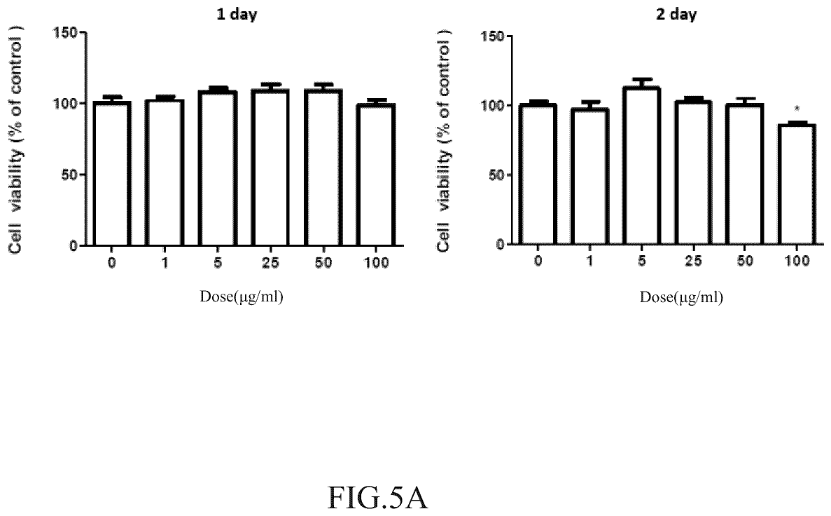

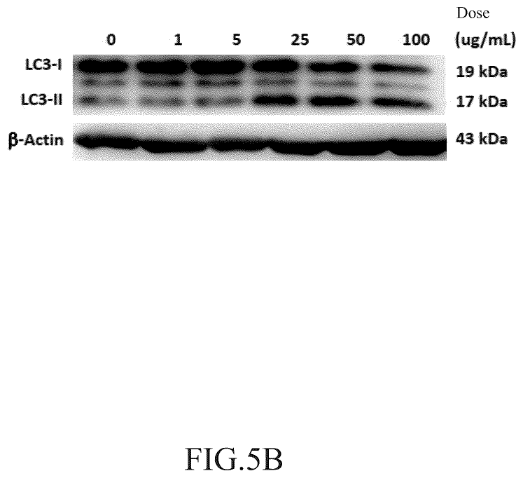

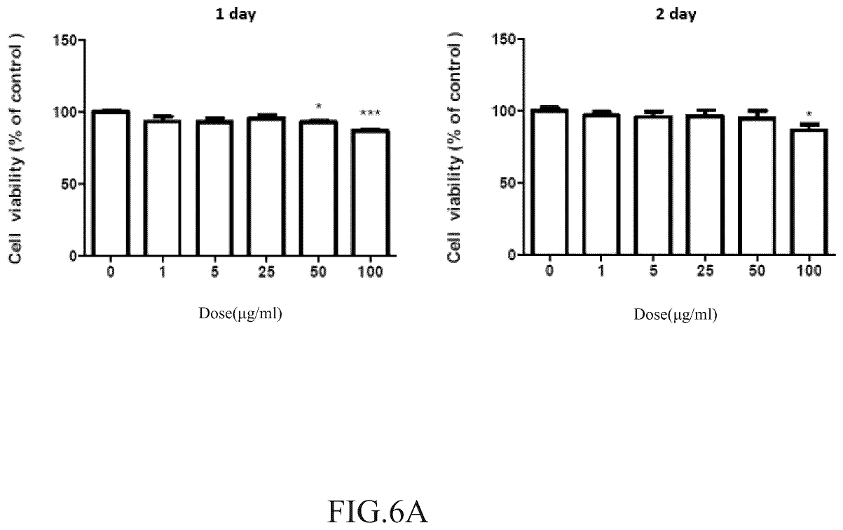

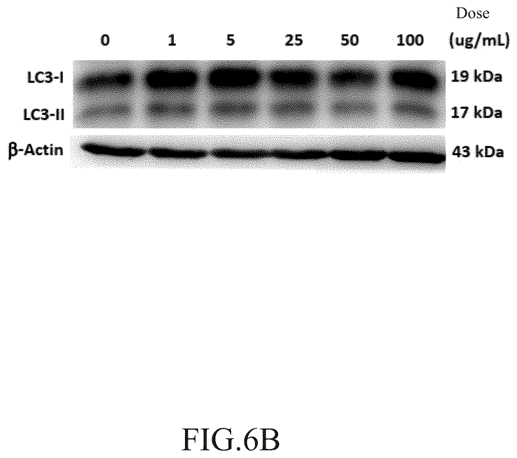

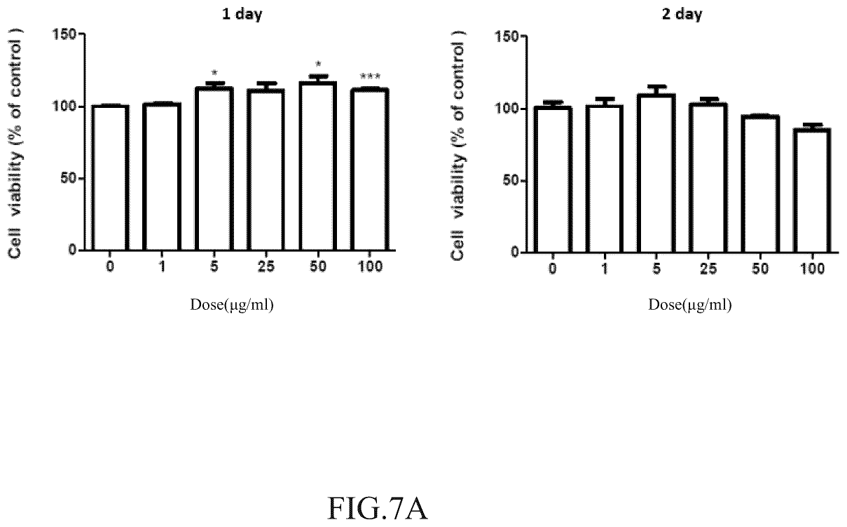

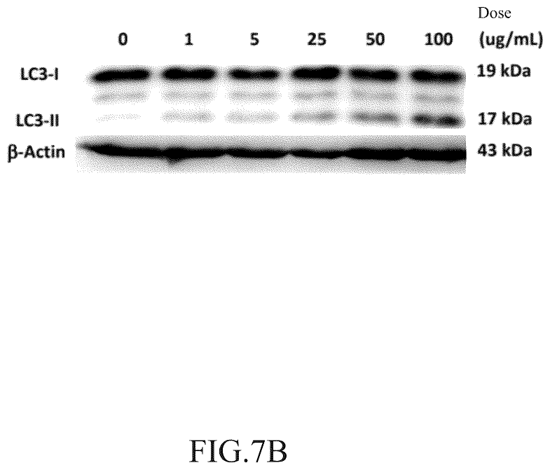

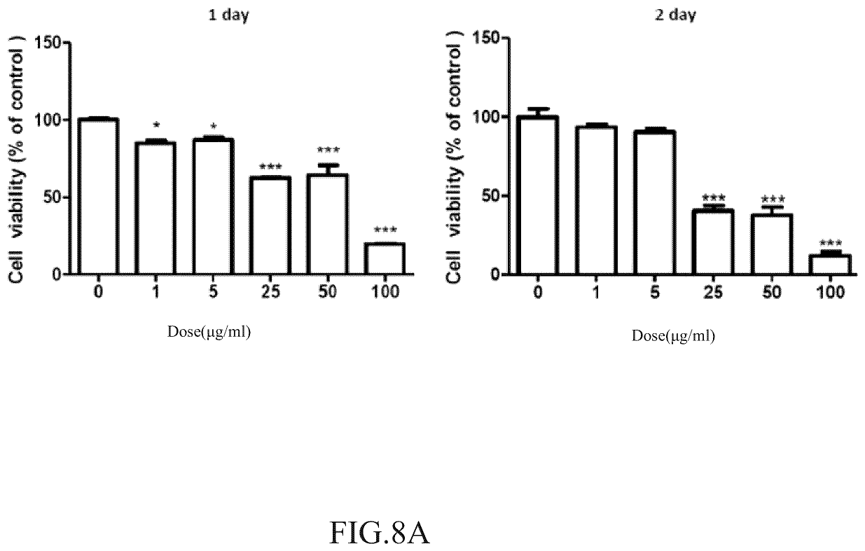

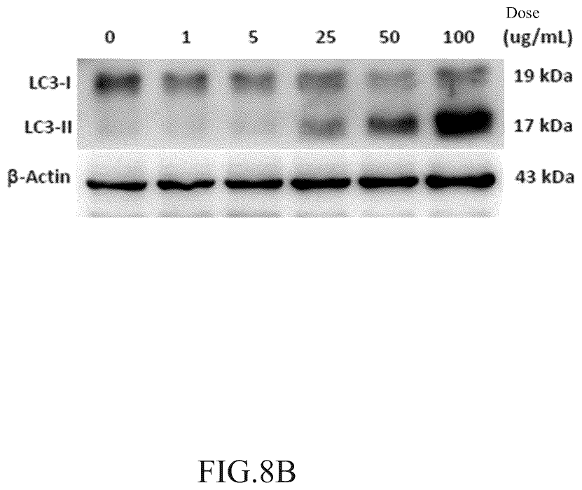

As illustrated in FIG. 5A, FIG. 6A, FIG. 7A, and FIG. 8A, FIG. 5A shows the SCLC cells (H1355) viability on the first day and the second day after treated by the material of the comparative example 1 at different effective doses, including 0 μg/ml, 1 μg/ml, 5 μg/ml, 25 μg/ml, 50 μg/ml, and 100 μg/ml. FIG. 6A shows the SCLC cells (H1355) viability on the first day and the second day after treated by the material of the comparative example 2 at different effective doses, including 0 μg/ml, 1 μg/ml, 5 μg/ml, 25 μg/ml, 50 μg/ml, and 100 μg/ml. FIG. 7A shows the SCLC cells (H1355) viability on the first day and the second day after treated by the material of the comparative example 3 at different effective doses, including 0 μg/ml, 1 μg/ml, 5 μg/ml, 25 μg/ml, 50 μg/ml, and 100 μg/ml. FIG. 8A shows the SCLC cells (H1355) viability on the first day and the second day after treated by the material of the experimental example at different effective doses, including 0 μg/ml, 1 μg/ml, 5 μg/ml, 25 μg/ml, 50 μg/ml, and 100 μg/ml.

In FIG. 5A, FIG. 6A, FIG. 7A, and FIG. 8A, the Standard Error (SE) of the test result of the comparative examples 1-3 and the experimental example is shown. The test result is obtained from three independent cellular experiments (n=3), wherein “*” indicates statistical significance (P<0.05) in the comparison of the test result of each of the comparative examples 1-3 and the experimental example.

As shown in FIG. 5A, in the result of either the first day or the second day of the comparative example 1, the cell viabilities of SCLC cells (H1355) treated by the material of the comparative example 1 at various effective doses do not significantly reduced. In other words, merely using Scutellaria baicalensis could not inhibit or reduce the viability or amount of SCLC cells (H1355).

As shown in FIG. 6A, in the result of either the first day or the second day of the comparative example 2, the cell viabilities of SCLC cells (H1355) treated by the material of the comparative example 2 at various effective doses do not significantly reduced. In other words, merely using Angelica dahurica could not inhibit or reduce the viability or amount of SCLC cells (H1355).

As shown in FIG. 7A, in the result of either the first day or the second day of the comparative example 3, the cell viabilities of SCLC cells (H1355) treated by the material of the comparative example 3 at various effective doses do not significantly reduced. In other words, using the mixture of Scutellaria baicalensis and Angelica dahurica could not inhibit or reduce the viability or amount of SCLC cells (H1355).

As shown in FIG. 8, in the result of the first day of the experimental example, the cell viabilities of SCLC cells (H1355) treated by the material of the experimental example at various effective doses, including 25 μg/ml, 50 μg/ml, and 100 μg/m, are significantly reduced to a range of approximately 20%-60%. Moreover, the cell viability of SCLC cells (H1355) treated by the material of the experimental example at effective dose of 100 μg/ml is significantly reduced. In the result of the second day of the experimental example, the cell viabilities of SCLC cells (H1355) treated by the material of the experimental example at various effective doses, including 25 μg/ml, 50 μg/ml, and 100 μg/m, are further reduced to a range of approximately 10%-50%. Similarly, the cell viability of SCLC cells (H1355) treated by the material of the experimental example at effective dose of 100 μg/ml is more significantly reduced. In other words, comparing to the comparative examples 1-3, the Chinese medicine composition of the experimental example could significantly and effectively inhibit the activation of SCLC cells (H1355) and reduce the amount of SCLC cells (H1355). Additionally, the Chinese medicine composition of experimental example at the effective doses of 25 μg/ml, 50 μg/ml, or 100 μg/ml could reduce the amount of SCLC cells (H1355) in the first day.

### 2. Autophage of SCLC Cells (H1355):

The LC3-I/LC3-II protein expression of SCLC cells (H1355) that is treated by each of the comparative examples 1-3 and the experimental example in various effective doses, including 1 μg/ml, 5 μg/ml, 25 μg/ml, 50 μg/ml, and 100 μg/ml, is analyzed. After SCLC cells (H1355) is treated by each of the comparative examples 1-3 and the experimental example, the LC3-I/LC3-II protein expression of lung adenocarcinoma cells (H460) in each example is detected through the electrophoresis.

As illustrated in FIG. 5B, FIG. 6B, FIG. 7B, and FIG. 8B, FIG. 5B shows the LC3-I/LC3-II protein electrophoresis diagram of SCLC cells (H1355) treated by the comparative example 1 at different effective doses, including 0 μg/ml, 1 μg/ml, 5 μg/ml, 25 μg/ml, 50 μg/ml, and 100 μg/m. FIG. 6B shows the LC3-I/LC3-II protein electrophoresis diagram of SCLC cells (H1355) treated by the comparative example 2 at different effective doses, including 0 μg/ml, 1 μg/ml, 5 μg/ml, 25 μg/ml, 50 μg/ml, and 100 μg/m. FIG. 7B shows the LC3-I/LC3-II protein electrophoresis diagram of SCLC cells (H1355) treated by the comparative example 3 at different effective doses, including 0 μg/ml, 1 μg/ml, 5 μg/ml, 25 μg/ml, 50 μg/ml, and 100 μg/m. FIG. 8B shows the LC3-I/LC3-II protein electrophoresis diagram of SCLC cells (H1355) treated by the experimental example at different effective doses, including 0 μg/ml, 1 μg/ml, 5 μg/ml, 25 μg/ml, 50 μg/ml, and 100 μg/m.

As illustrated in FIG. 5B, FIG. 6B, FIG. 7B, and FIG. 8B, the LC3-II protein expression of SCLC cells (H1355) treated by the experimental example at the effective doses, including 50 μg/ml and 100 μg/ml, is obviously greater than that is treated by the comparative examples 1-3. In other words, the Chinese medicine composition of the experimental example at the effective dose of 50 μg/ml or 100 μg/ml could induce the LC3-I/LC3-II protein expression in SCLC cells (H1355), leading to autophagy of SCLC cells (H1355), thereby inhibiting and reducing the activation of SCLC cells (H1355).

To sum up, the Chinese medicine composition of the current embodiment includes Angelica dahurica, Scutellaria baicalensis, Lepidium apetalum, apricot kernel, and mirabilite at a specific parts by weight. Through cellular experiment, comparing to Angelica dahurica, Scutellaria baicalensis, or the mixture of Angelica dahurica and Scutellaria baicalensis, the Chinese medicine composition at the specific effective dose could effectively inhibit the activation and reduce the amount of lung adenocarcinoma cells (H460) and SCLC cells (H1355) within a short time due to the pharmacological interactions between Angelica dahurica, Scutellaria baicalensis, Lepidium apetalum, apricot kernel, and mirabilite. The Chinese medicine composition at the effective doses of 25 μg/ml, 50 μg/ml, or 100 μg/ml could inhibit or reduce the activation of lung adenocarcinoma cells (H460) and SCLC cells (H1355). Preferably, the chinese medicine composition at the effective doses of 50 μg/ml or 100 μg/ml has the preferred therapeutic effect to lung cancer (i.e. effectively reduce the activation and amount of the cancer cells and induce the expression of LC3-I/LC3-II protein leading to autophagy).

It must be pointed out that the embodiment described above is only a preferred embodiment of the present invention. All equivalent formulas and methods which employ the concepts disclosed in this specification and the appended claims should fall within the scope of the present invention.

What is claimed is:

1. A Chinese medicine composition for treating lung cancer, comprising
Angelica dahurica ranging from 3 to 30 parts by weight;
Scutellaria baicalensis ranging from 3 to 30 parts by weight;
Lepidium apetalum ranging from 8 to 35 parts by weight;
apricot kernel ranging from 5 to 30 parts by weight; and
mirabilite ranging from 0.3 to 5 parts by weight.

2. The Chinese medicine composition as claimed in claim 1, wherein a sum of a content of Angelica dahurica and a content of the Scutellaria baicalensis is a first portion; a sum of a content of Lepidium apetalum, a content of apricot kernel, and a content of mirabilite is a second portion; the first portion is greater than the second portion.

3. The Chinese medicine composition as claimed in claim 2, wherein Angelica dahurica ranges from 5 to 10 parts by weight; Scutellaria baicalensis ranges from 10 to 15 parts by weight; Lepidium apetalum ranges from 10 to 15 parts by weight; apricot kernel ranges from 8 to 12 parts by weight; and mirabilite ranges from 0.5 to 1.5 parts by weight.

4. The Chinese medicine composition as claimed in claim 3, wherein Angelica dahurica ranges from 8 to 10 parts by weight; Scutellaria baicalensis ranges from 13 to 15 parts by weight; Lepidium apetalum ranges from 10 to 12 parts by weight; apricot kernel ranges from 8 to 10 parts by weight; and mirabilite ranges from 1 to 1.5 parts by weight.

5. The Chinese medicine composition as claimed in claim 1, wherein a sum of a content of Scutellaria baicalensis and a content of the Lepidium apetalum is a third portion; a sum of a content of Angelica dahurica, a content of apricot kernel, and a content of mirabilite is a fourth portion; the third portion is greater than the fourth portion.

6. The Chinese medicine composition as claimed in claim 5, wherein Angelica dahurica ranges from 5 to 10 parts by weight, Scutellaria baicalensis ranges from 20 to 30 parts by weight, Lepidium apetalum ranges from 20 to 30 parts by weight, apricot kernel ranges from 8 to 12 parts by weight, and mirabilite ranges from 0.5 to 1.5 parts by weight.

7. The Chinese medicine composition as claimed in claim 1, wherein a sum of a content of Angelica dahurica and a content of the Scutellaria baicalensis is a first portion; a sum of a content of Lepidium apetalum, a content of apricot kernel, and a content of mirabilite is a second portion; the second portion is greater than the first portion.

8. The Chinese medicine composition as claimed in claim 7, wherein Angelica dahurica ranges from 15 to 25 parts by weight, Scutellaria baicalensis ranges from 10 to 15 parts by weight, Lepidium apetalum ranges from 20 to 30 parts by weight, apricot kernel ranges from 15 to 25 parts by weight, and mirabilite ranges from 2 to 4 parts by weight.

9. An usage of the Chinese medicine composition as claimed in claim 1 for inhibiting or reducing an activation of lung cancer cells, wherein when the Chinese medicine composition at an effective dose of 1 μg/ml to 100 μg/ml administrates to lung cancer cells, the LC3-I/LC3-II protein is induced to express, leading to autophage of lung cancer cells.

10. The usage of the Chinese medicine composition as claimed in claim 9, wherein the effective dose of the Chinese medicine composition is in a range of 25 μg/ml to 100 μg/ml, and the lung cancer cells include Non-Small Cell Lung Cancer (NSCLC) cells and/or Small Cell Lung Cancer (SCLC) cells.
